(19) United States
(12) Patent Application Publication
(10) Pub. No.: US 2026/0266793 A1
(43) Pub. Date: Sep. 10, 2026

(54) SYSTEMS AND METHODS FOR DETERMINING FLUID AGING

(71) Applicant: Simple Labs, Inc., Calistoga, CA (US)

(72) Inventor: Mike Slone, Calistoga, CA (US)

(21) Appl. No.: 19/458,887

(22) Filed: Jan. 25, 2026

Related U.S. Application Data
(60) Provisional application No. 63/749,891, filed on Jan. 27, 2025.

(51) Int. Cl. G01N 33/14; G01N 21/01; G01N 21/3577

(52) U.S. Cl. CPC G01N 33/146; G01N 21/01; G01N 21/3577; G01N 2021/0168

(57) ABSTRACT

Embodiments of the present disclosure disclose methods, systems, and a sensing device for determining fluid aging. The sensing device includes housing, at least one radiating source, at least one sensor, and control circuitry communicably coupled to the at least one radiating source and the at least one sensor. The at least one radiating source is configured to emit radiating light of a plurality of wavelengths through a portion of the fluid. Further, at least one sensor is configured to receive the radiated light upon interacting with the portion of the fluid and generate an output signal. Furthermore, the control circuitry is configured to receive the output signal from the at least one sensor and determine an absorption spectrum associated with the portion of the fluid based at least on the output signal. Furthermore, the control circuitry is configured to compute an absorption value associated with the portion of the fluid.

### TECHNICAL FIELD

The present invention relates to electronic systems for determining fluid aging, and more particularly relates to systems and methods for determining fluid (e.g., wine, bourbon, etc.) aging based on absorption spectral analysis.

### BACKGROUND

Alcoholic beverages such as wine, bourbon, beer, rum, whisky, and the like require storage in a barrel (or a container) for an extended period of time during their production. Typically, the aging of the alcohol beverages is estimated based on a predefined timeline (e.g., 2 years for bourbon, decades for certain wines, etc.). However, some containers may not be fully airtight, whether by design or due to limitations, leading to potential loss of liquid (e.g., the alcohol beverages) through evaporation, leakage, or other means, which can reduce the volume over time. For example, wooden barrels containing the bourbon may evaporate naturally over time, which is a necessary part of the distillation process. To monitor the aging of the alcohol beverages, conventional techniques such as visual inspection may be implemented to monitor the transition in hue (e.g., bourbon darkens from clear to amber, wine changes from various shades of red and yellow with age), determine residue or sediment buildup in the alcohol beverages, and the like. However, the conventional techniques that involve manual intervention lack precision and are influenced by lighting and human perception. Further, variations in human perception and environmental factors may lead to inaccurate results. Furthermore, the conventional techniques require significant time and expertise, making it unsuitable for rapid or automated analysis.

In recent times, Internet-based computing networks used in combination with wireless sensors allow users to access real-time data on wireless devices. One such example is sensor devices used for determining aging of the alcohol beverages. The sensor devices may be used in modern techniques to monitor the aging of the alcohol beverages. Although, the sensor devices determine the aging of the alcohol beverages in the modern techniques, the sensor devices may fail to provide real-time changes occurring in the containers storing the alcohol beverages. Furthermore, some of the modern techniques often require physical samples of the alcohol beverages, thus leading to a decrease in the volume of the alcoholic beverages. Additionally, environmental factors such as extreme temperature changes and high humidity or condensation may affect sensor performance and accuracy, resulting in false readings and cause malfunctions. Also, there is a tremendous amount of time and labor involved in randomly collecting samples from numerous barrels in various locations.

Therefore, there is a need for systems and methods for determining aging of the alcohol beverages that overcome the aforementioned deficiencies along with providing other advantages.

### SUMMARY

Various embodiments of the present disclosure disclose methods and systems for determining the aging of a portion of a fluid (e.g., wine, bourbon, etc.) stored in receptacles.

In an embodiment, a sensing device is disclosed. The sensing device includes a housing, at least one radiating source, at least one sensor, and control circuitry communicably coupled to the at least one radiating source and the at least one sensor. The at least one radiating source is disposed within the housing. The at least one radiating source is configured to emit radiating light of a plurality of wavelengths through a portion of the fluid stored in a receptacle. Further, at least one sensor is configured to receive the radiated light upon interacting with the portion of the fluid in the receptacle and generate an output signal proportional to the amount of the radiating light being absorbed by the portion of the fluid. Furthermore, the control circuitry is configured at least in part to receive the output signal from the at least one sensor and determine an absorption spectrum associated with the portion of the fluid based at least on the output signal. Furthermore, the control circuitry is configured to compute an absorption value associated with the portion of the fluid based at least on the absorption spectrum and the output signal.

In another embodiment, a method performed by a sensing device is disclosed. The method includes receiving radiated light upon interacting with a portion of a fluid stored in a receptacle and generating an output signal proportional to an amount of the radiating light being absorbed by the portion of the fluid. Further, the method includes determining an absorption spectrum associated with the portion of the fluid based at least on the output signal. Furthermore, the method includes computing an absorption value associated with the portion of the fluid based at least on the absorption spectrum and the output signal.

In yet another embodiment, a system is disclosed. The system includes a receptacle configured to store a portion of a fluid and a sensing device disposed within the receptacle. The sensing device includes a housing and at least one radiating light source disposed within the housing. The at least one radiating light source is configured to emit radiating light of a plurality of wavelengths through the portion of the fluid in the receptacle. Further, the sensing device includes at least one sensor configured to receive the radiated light upon interacting with the portion of the fluid in the receptacle and generate an output signal proportional to the amount of the radiating light being absorbed by the portion of the fluid. Furthermore, the sensing device includes a control circuitry communicably coupled to the at least one sensor and the at least one radiating light source. The control circuitry is configured at least in part to receive the output signal from the at least one sensor and determine an absorption spectrum associated with the portion of the fluid based at least on the output signal. Further, the control circuitry is configured to compute an absorption value associated with the portion of the fluid based at least on the absorption spectrum and the output signal.

### BRIEF DESCRIPTION OF THE FIGURES

The following detailed description of illustrative embodiments is better understood when read in conjunction with the appended drawings. For the purposes of illustrating the present disclosure, exemplary constructions of the disclosure are shown in the drawings. However, the present disclosure is not limited to a specific device, or a tool and instrumentalities disclosed herein. Moreover, those in the art will understand that the drawings are not to scale. Wherever possible, like elements have been indicated by identical numbers:

FIG. 1 illustrates an example representation of an environment related to at least some example embodiments of the present disclosure;

FIG. 2 illustrates a schematic representation of a sensing device being inserted into a receptacle for determining the aging of fluid in the receptacle, in accordance with an embodiment of the present disclosure;

FIG. 3 illustrates a graphical representation of an absorption spectrum of fluid samples, in accordance with an embodiment of the present disclosure;

FIG. 4 illustrates a simplified block representation of electronic circuitry, in accordance with an embodiment of the present disclosure; and

FIG. 5 illustrates a flow diagram of a method for determining the aging of the portion of the fluid, in accordance with the embodiments of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features.

Assessing the age of fluids, such as bourbon, plays a crucial role in confirming authenticity, meeting legal requirements, and refining taste characteristics through chemical shifts that occur during the barrel-aging process. The proposed system and method employ absorption spectroscopy to assess the concentration of alcohol content, determine aging, and color of a portion of a fluid (hereinafter the terms “portion of the fluid” and “fluid” are used interchangeably). The system includes a receptacle to store the portion of the fluid and a sensing device. The sensing device includes a housing that includes a base structure, a left support structure, and a right support structure. The right support structure and the left support structure extend from the base structure and are configured to enclose at least one radiating light source and at least one sensor, respectively. The left support structure and the right support structure are positioned at a distance. The distance defines a path length, in other words, the distance denotes the effective length that the radiating light travels in the portion of the fluid. The at least one sensor receives the radiated light upon interacting with the portion of the fluid in the receptacle and generates an output signal proportional to the amount of the radiating light being absorbed by the portion of the fluid. Further control circuitry associated with the sensing device is configured to determine an absorption spectrum associated with the portion of the fluid based at least on the output signal. Based on the absorption spectrum and the output signal, the control circuitry determines an absorption value that is indicative of concentration of alcohol in the portion of the fluid.

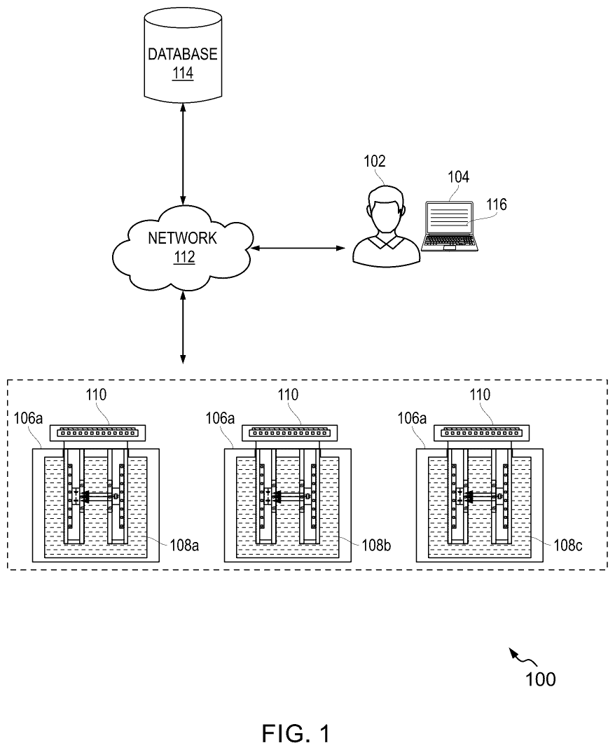

Various embodiments of the present invention are described hereinafter with reference to FIG. 1 to FIG. 5.

FIG. 1 illustrates an example representation of an environment 100 related to at least some example embodiments of the present disclosure. Although the environment 100 is presented in one arrangement, other arrangements are also possible where the parts of the environment 100 (or other parts) are arranged or interconnected differently. The environment 100 corresponds to a system for determining aging of fluid. In one example, the system disclosed in the environment 100 may be configured to determine the aging of fluid such as alcoholic beverages (e.g., wine, bourbon, and the like).

The environment 100 includes a user 102 associated with a user device 104. The user device 104 may include at least a laptop computer, a phablet computer, a handheld personal computer, a Virtual Reality (VR) device, a netbook, a Web book, a tablet computing device, a smartphone, or other mobile computing devices. Further, the environment 100 includes a plurality of receptacles, such as a receptacle 106a, a receptacle 106b, and a receptacle 106c. Each of the receptacles 106a, 106b, and 106c may be configured to store fluid such as fluid 108a, fluid 108b, and fluid 108c, respectively. For example, the fluid 108a stored in the receptacle 106a may be wine, and the fluids 108b-108c stored in the respective receptacles 106b-106c may be bourbon. Further, each of the receptacles 106a-106c is equipped with a sensing device 110. The sensing device 110 is inserted into the receptacles 106a-106c storing the corresponding fluids 108a-108c via an aperture (not shown in FIG. 1) configured in the receptacles 106a-106c. The sensing device 110 may be configured to determine the aging of the fluids 108a-108c which will be explained further in detail.

Various entities in the environment 100 may connect to a network 112 in accordance with various wired and wireless communication protocols, such as Transmission Control Protocol and Internet Protocol (TCP/IP), User Datagram Protocol (UDP), 2nd Generation (2G), 3rd Generation (3G), 4th Generation (4G), 5th Generation (5G) communication protocols, Long Term Evolution (LTE) communication protocols, Long Range (LoRa) Gateway Protocol or any combination thereof. In some instances, the network 112 may include a secure protocol (e.g., Hypertext Transfer Protocol (HTTP)), and/or any other protocol, or set of protocols. In an example embodiment, the network 112 may include, without limitation, a local area network (LAN), a wide area network (WAN) (e.g., the Internet), a mobile network, a virtual network, and/or another suitable public and/or private network capable of supporting communication among two or more of the entities illustrated in FIG. 1, or any combination thereof.

In an embodiment, the user 102 may be an individual associated with managing the fluids 108a-108c in the corresponding receptacles 106a-106c. In another embodiment, the user 102 may be a worker or a technician in a winery production industry and is associated with tracking and monitoring the aging of the fluids 108a-108c in the corresponding receptacles 106a-106c.

The sensing device 110 may be configured to host and manage an application 116. The application 116 is a set of computer-executable codes configured to allow the user 102 to track and/or visualize the aging of the fluids 108a-108c stored in the corresponding receptacles 106a-106c. In one embodiment, the application 116 may be accessed as a web-based application on the user device 104. In another embodiment, the user device 104 may access an instance of the application 116 for installation on the user device 104 using application stores (not shown in FIGS.) associated with operating systems such as Apple iOS®, Android™ OS, Google Chrome OS, Symbian OS®, Windows Mobile® OS, and the like.

The sensing device 110 may include electronics (not shown in FIG. 1) configured to monitor the aging of the fluids 108a-108c. In particular, the sensing device 110 includes the electronics (such as at least one circuitry, or processor(s)) to perform absorbance spectroscopy. This enables the measurement of light absorption by a fluid (e.g., the fluids 108a-108c) across a broad spectrum of wavelengths. The electronics of the sensing device 110 may utilize at least one radiating light source (e.g., narrowband light emitting diodes (LEDs)) of various wavelengths, which sequentially emit radiating light through the fluid sample (such as the fluids 108a-108c) (not shown in FIG. 1). The at least one radiating light source (hereinafter interchangeably referred to as ‘the radiating light source) is detected by at least one sensor (not shown in FIG. 1) associated with the sensing device 110. The at least one sensor of the sensing device 110 may convert the received radiating light source into electric pulses. The electric pulses are subsequently amplified, filtered, and measured by the sensing device 110 for precise light measurements and to determine the aging of the fluids 108a-108c stored in the corresponding receptacles 106a-106c. Further, the sensing device 110 may be configured to transfer the determined data (i.e., the data related to the aging of the fluids 108a-108c) to the user 102 via the application 116. The data related to the aging of the fluids 108a-108c may be stored in a database 114. Thus, this approach provides real-time testing of the fluid 108a-108c without manual intervention, enables continuous monitoring of the fluids 108a-108c in the corresponding receptacles 106a-106c, provides immediate insights into the fluids 108a-108c to monitor threats throughout the life cycle, and results in improved tracking.

The number and arrangement of systems, devices, and/or networks shown in FIG. 1 are provided as an example. There may be other systems, devices, and/or networks; fewer systems, devices, and/or networks; different systems, devices, and/or networks, and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices.

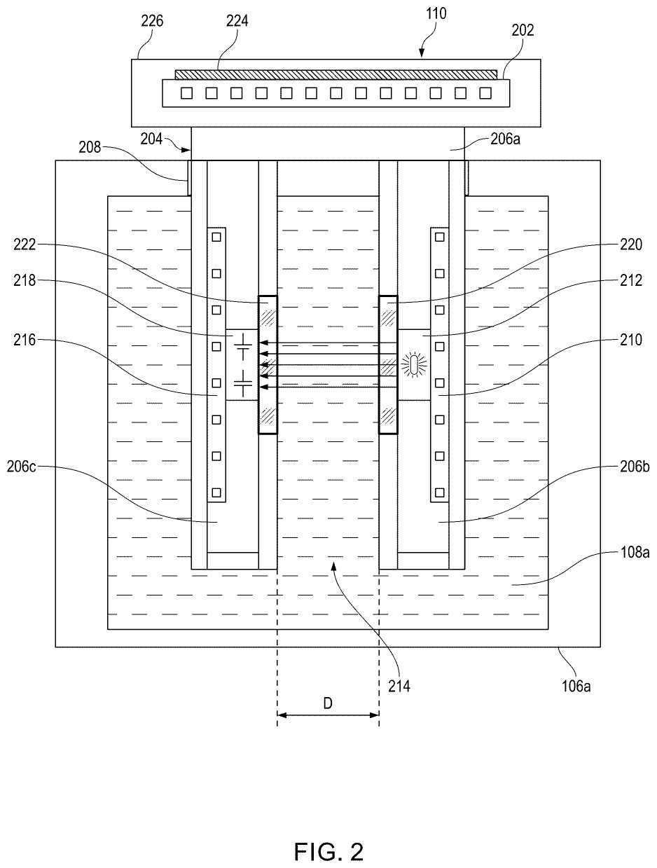

FIG. 2 illustrates a schematic representation of the sensing device 110 being inserted into a receptacle for determining the aging of fluid in the receptacle, in accordance with an embodiment of the present disclosure. The present disclosure is explained with reference to determining the aging of the fluid 108a stored in the receptacle 106a using the sensing device 110. It should be understood that one or more operations performed by the sensing device 110 for determining the aging of the fluid 108a may be applied for determining the aging of the fluids 108b-108c.

The sensing device 110 includes a control circuitry 202 and a housing 204. The housing 204 provides support to one or more components of the sensing device 110. In particular, the housing 204 includes a base structure 206a, a right support structure 206b, and a left support structure 206c. The right support structure 206b and the left support structure 206c extend from the base structure 206a. The right support structure206b and the left support structure 206c are configured at a distance (D). The distance (D) between the right support structure 206b and the left support structure 206c is a critical parameter in the absorption spectroscopy that significantly impacts the performance of the sensing device 110 and the accuracy of the measurement, which will be explained further. Further, the housing 204, including the base structure 206a, the right support structure 206b, and the left support structure 206c, conforms to a U-shaped structure.

Furthermore, the control circuitry 202 may be equipped in an enclosure 226. The enclosure 226 is removably secured to the housing 204 (i.e., the base structure 206a). The sensing device 110 further includes a power source 224. The power source 224 may be disposed in the enclosure 226. The power source 224 may provide a power supply to at least the control circuitry 202 and one or more components (such as a first control unit 210, a second control unit 216, at least one radiating light source 212, at least one sensor 218, and the like) of the sensing device 110. The power source 224 may provide one of an alternating current output or a direct current output. In an embodiment, the power source 224 includes a direct current power source, such as a rechargeable battery (e.g., a lithium-ion battery), operable to provide the required electrical power for the operation of the sensing device 110. Further, the power source 224 may include electrical and/or electronic components or circuits for enabling the use of wired or wireless charging. Alternatively, the power source 224 may include electrical and/or electronic components or circuits for enabling the use of alternating current to provide the required electrical power for the operation of the sensing device 110. Further, the sensing device 110 may include a charging port (not shown in FIGS.) to plug an electric line for receiving electric power for charging the power source 224.

As shown, the sensing device 110 is inserted into the receptacle 106a storing the fluid 108a via an aperture 208 configured in the receptacle 106a. The base structure 206a and the enclosure 226 of the housing 204 are positioned outside of the receptacle 106a, while the right support structure 206b and the left support structure 206c are disposed in the receptacle 106a. The right support structure 206b and the left support structure 206c, disposed in the receptacle 106a, are in contact with the fluid 108a stored in the receptacle 106a. The housing 204 (i.e., the base structure 206a, the right support structure 206b, and the left support structure 206c) may be made using food-grade materials, for example, steel, aluminum, or any other materials as per the design feasibility and requirements.

The sensing device 110 includes the first control unit 210. The first control unit 210 may be disposed in the right support structure 206b. The first control unit 210 may include at least one processor, such as a processor and memory devices. The memory devices may store machine-executable instructions. Further, the at least one processor may be capable of executing the machine-executable instructions to perform one or more operations described herein.

Further, the sensing device 110 includes the at least one radiating light source 212. The at least one radiating light source 212 is disposed in the right support structure 206b. The at least one radiating light source 212 is communicably coupled to the first control unit 210. In other words, the at least one radiating light source 212 may be configured in the first control unit 210. The first control unit 210 may be configured to provide a power supply to the at least one radiating source 212. In an embodiment, the control circuitry 202 may provide a signal to the first control unit 210 for operating the at least one radiating light source 212. In another embodiment, the first control unit 210 may be pre-configured to operate the at least one radiating light source 212 to determine one or more attributes related to the fluid 108a. The one or more attributes related to the fluid 108a may include the aging of the fluid 108a, concentration of alcohol content in the fluid 108a, and the like.

The at least one radiating light source 212 may include Light Emitting Diodes (LEDs) configured to emit the radiating light of a broad spectrum of wavelengths (e.g., between the visible light region and the infrared region). The at least one radiating light source 212 may include narrowband LEDs of various wavelengths that sequentially emit light through a portion of the fluid 108a accumulated in a region 214 between the right support structure 206b and the left support structure 206c. For example, the at least one radiating light source 212 may include five narrowband LEDs, where three LEDs may be configured to emit the radiating light between 400 nanometers (nm) and 550 nm to measure the changes in the fluid 108a and two LEDs may be configured to emit the radiating light between 905 nm and 980 nm to determine the concentration of the alcohol content in the fluid 108a. Alternatively, the at least one radiating light source 212 may include, but not limited to, Tungsten-Halogen Lamps, Laser Diodes, deuterium discharge lamps, and the like.

Parameters associated with the at least one radiating light source 212, such as radiating light intensity, spectral distribution, detector responsivity, distance (path length), alignment, and environmental conditions, introduce deterministic and systematic deviations between the actual optical measurements and the measured signal. Hence, the sensing device 110 is calibrated with device-specific calibrated values. The calibration values define the device-specific proportionality between measured absorbance and alcohol concentration under fixed experimental conditions. These values, obtained from a calibration curve using certified reference standards, incorporate the effective molar absorptivity, optical path length, wavelength accuracy, at least one sensor (e.g., photo detector) response, and baseline corrections. The calibration values compensate for non-ideal behavior of the at least one radiating light source 212, such as stray light, lamp intensity fluctuations, and detector nonlinearity. The calibration values enable accurate quantification of unknown samples, validation of Beer-Lambert law linearity within the working range, assessment of sensitivity and limit of detection, and ensure traceability, reproducibility, and analytical reliability of absorption spectroscopic measurements.

In an embodiment, the calibration values may include at least one sensor response (e.g., photo detector response) to the radiating light in the near-infrared region and visible region in a hundred percent alcohol solution. The sensor response in the near-infrared region and the visible region, combined with the temperature of the hundred percent alcohol solution, may be provided to the user (say, the user 102). Further, the sensing device 110 may be associated with a memory (not shown in the figure) configured to store the configuration values.

Further, the sensing device 110 includes the second control unit 216. The second control unit 216 may be disposed in the left support structure 206c. The second control unit 216 may include at least one processor, such as a processor and memory devices. The memory devices may store machine-executable instructions. Further, the at least one processor may be capable of executing the machine-executable instructions to perform one or more operations described herein.

The sensing device 110 includes the at least one sensor 218. The at least one sensor 218 may be communicably coupled to the second control unit 216. In other words, the at least one sensor 218 may be configured on the second control unit 216. The second control unit 216 may be configured to provide a power supply for operating the at least one sensor 218. As explained above, the at least one radiating light source 212 is configured to emit the radiating light of different wavelengths sequentially. The radiating light emitted by the at least one radiating light source 212 is transmitted through a first window 220 of the right support structure 206b, and interacts with the portion of the fluid 108a accumulated in the region 214. In particular, the radiating light passing through the first window 220 interacts with the molecules of the fluid 108a. Typically, specific wavelengths of the radiating light emitted by the radiating light source 212 are absorbed by different molecular bonds (such as O—H, C—H, and N—H bonds) of the fluid 108a (e.g., wine) present in the region 214. The radiating light, upon interacting with the portion of the fluid 108a in the region 214 is received by the at least one sensor 218 through a second window 222 configured in the left support structure 206c.

It is to be noted that the first window 220 and the second window 222 may be configured using transparent materials or any other materials that do not affect the optical properties of the radiating light being emitted by the at least one radiating light source 212 and the radiating light being received by the at least one sensor 218 upon interacting with the portion of the fluid 108a. In other words, the first window 220 and the second window 222 may be referred to as optical windows. As shown, the first window 220 is configured in-line with the second window 222 for effective transmission of the radiating light from the at least one radiating light source 212 to the at least one sensor 218. In an embodiment, the radiating light emanated from the at least one radiating light source 212 may be reflected onto the at least one sensor 218. As explained above, the distance (D) between the at least one radiating light source 212 and the at least one sensor 218 is related to the path length of the radiating light traveling through the sample (i.e., the fluid 108a). A proper distance ensures that the at least one sensor 218 receives sufficient intensity of the radiating light after passing through the sample (i.e., the portion of the fluid 108a). In one example scenario, if the distance (DO is too short, the radiating light may not interact sufficiently with the portion of the fluid 108a, leading to weak absorbance signals. In another example scenario, if the distance (D) is too long, the intensity of the radiating light may drop below the detection threshold due to scattering, absorption, or divergence, reducing signal to noise ratio (SNR).

The at least one sensor 218 may be configured to generate an electrical signal based on the radiating light being received by the at least one sensor 218 upon interacting with the portion of the fluid 108a present in the region 214. For example, the at least one sensor 218 may be a color sensor including color-sensitive filters, amplifiers, and sensor arrays for sensing a wide variety of colors. The at least one sensor 218 may be configured to compute a weightage for each of the primary colors in the radiating light received at the at least one sensor 218 upon interacting with the portion of the fluid 108a. Some non-exhaustive examples of the color sensor (i.e. the at least one sensor 218) include TCS 3200, color PAL, TCS 3400, TCS 34715, TCS 34727, SEN-11195, Lego Mindstorms EV3, and the like. Further, the at least one sensor 218 may generate an output signal (i.e., the electrical signal) proportional to the amount of the radiating light being absorbed by the portion of the fluid 108a. In addition, the at least one sensor 218 may compute an absorption value based on the amount of the radiating light being absorbed by the portion of the fluid 108a.

Thereafter, the second control unit 216 may be configured to amplify the output signal, generating an amplified output signal, using operational amplifiers for further processing (such as digitization). The amplified output signal may be a continuous analog signal. Further, the second control unit 216 may convert the amplified output signal into discrete digital values (i.e., digitized data) by sampling the amplified output signal at regular intervals and quantizing it into binary numbers. Further, the digitized data (i.e., the discrete digital values) is in the form of a numerical array that represents intensity levels of the radiating light at specific time intervals or wavelengths. Furthermore, the second control unit 216 transmits the digitized data to the control circuitry 202 for further analysis. The control circuitry 202 may transmit the digitized data to the user 102 via the network 112. The digitized data may represent absorbance across a range of wavelengths (spectrum). For instance, peaks and valleys in the spectrum indicate the presence of specific compounds or aging markers. In one example, changes in red-to-brown hues (absorbance at 420 nm, 520 nm, and 620 nm) are indicative of the aging stages of the wine (or the fluid 108a). In another example, darkening due to wood-extracted compounds like lignin's and vanillin may be tracked by measuring absorption in the visible spectrum for the bourbon (e.g., the fluids 108b-108c). Further, absorption in the UV-visible range (e.g., 280 nm for tannins and phenols) indicates the concentration and evolution of phenolic compounds that are crucial for the flavor and color of the fluids 108a-108c.

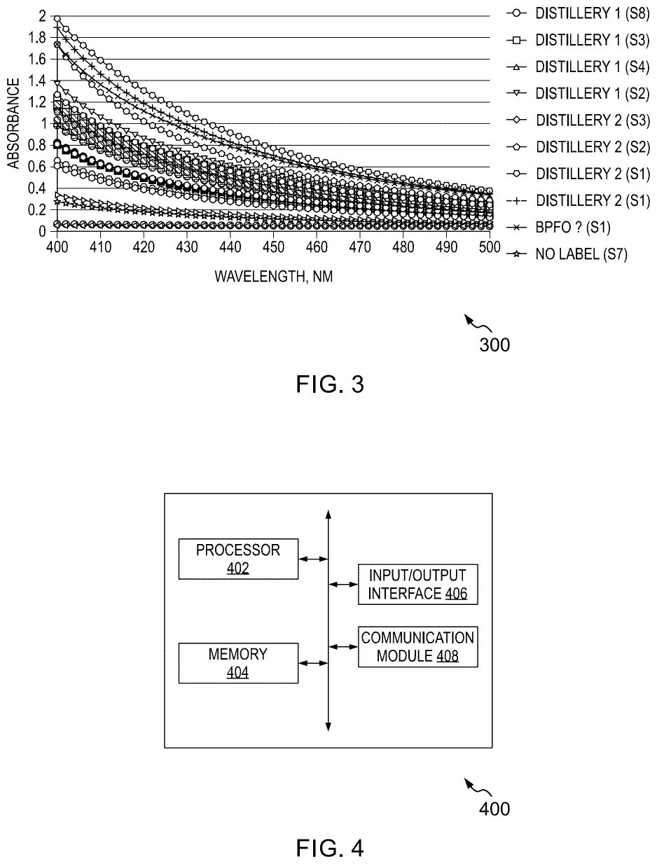

FIG. 3 illustrates a graphical representation 300 of an absorption spectrum of fluid samples, in accordance with an embodiment of the present disclosure. The absorption spectrum may be generated for sample fluids (exemplarily represented as S1, S2, S3, S4, S7, S8) in the wavelength range of 400 nm to 500 nm. For illustration purposes, different symbols are used to represent the absorption spectrum of each sample (S1, S2, S3, S4, S7, S8) in the graphical representation 300. For example, the same fluids (S1, S2, S3, S4, S7, S8) may be the fluids 108a-108c.

Furthermore, FIG. 3 illustrates a plurality of curves that may be obtained by plotting absorbance across the plurality of wavelengths of the radiating light. The plurality of curves indicates the rate of aging of the portion of the fluid. In other words, the plurality of curves determines the amount of interaction between the portion of the fluid and a charred layer of wood on the inside of the barrel used for storing the portion of the fluid, over time. The amount of interaction varies across barrels.

FIG. 4 illustrates a simplified block representation of electronic circuitry 400, in accordance with an embodiment of the present disclosure. The electronic circuitry 400 is an example of the control circuitry 202, the first control unit 210, and the second control unit 216 of the sensing device 110.

The electronic circuitry 400 includes at least one processor, such as a processor 402 and a memory 404. It is noted that although the electronic circuitry 400 is depicted to include only one processor, the electronic circuitry 400 may include more processors therein. In an embodiment, the memory 404 is capable of storing machine-executable instructions. Further, the processor 402 is capable of executing the machine-executable instructions to perform one or more operations described herein. In an embodiment, the processor 402 may be embodied as a multi-core processor, a single-core processor, or a combination of one or more multi-core processors and one or more single-core processors. For example, the processor 402 may be embodied as one or more of various processing devices, such as a coprocessor, a microprocessor, a controller, a Digital Signal Processor (DSP), a processing circuitry with or without an accompanying DSP, or various other processing devices including integrated circuits such as, for example, an Application Specific Integrated Circuit (ASIC), a Field Programmable Gate Array (FPGA), a microcontroller unit (MCU), a hardware accelerator, a special-purpose computer chip, or the like. In an embodiment, the processor 402 may be configured to execute hard-coded functionality. In an embodiment, the processor 402 is embodied as an executor of software instructions, wherein the instructions may specifically configure the processor 402 to perform the algorithms and/or operations described herein when the instructions are executed.

The memory 404 may be embodied as one or more volatile memory devices, one or more non-volatile memory devices, and/or a combination of one or more volatile memory devices and non-volatile memory devices. For example, the memory 404 may be embodied as semiconductor memories (such as mask (ROM), programmable ROM (PROM, Erasable PROM (EPROM), flash memory, Random Access Memory (RAM), etc.), magnetic storage devices (such as hard disk drives, floppy disks, magnetic tapes, etc.), optical magnetic storage devices (e.g., magneto-optical disks), Compact Disc Read Only Memory (CD-ROM), Compact Disc Recordable (CD-R), Compact Disc Rewritable (CD-R/W), Digital Versatile Disc (DVD) and BLU-RAY® Disc (BD).

The electronic circuitry 400 further includes an Input/Output (I/O) module 406 (hereinafter referred to as an ‘I/O module 406’) and at least one communication module, such as a communication module 408. In an embodiment, the I/O module 406may include mechanisms configured to receive inputs (or data from the at least one sensor 218) and provide outputs to the user 102.

In an embodiment, the processor 402 may include I/O circuitry configured to control at least some functions of one or more elements of the I/O module 406, such as, for example, a speaker, a microphone, a display, and/or the like. The processor 402 and/or the I/O circuitry may be configured to control one or more functions of the one or more elements of the I/O module 406 through computer program instructions, for example, software and/or firmware, stored on a memory, for example, the memory 404, and/or the like, accessible to the processor 402.

The communication module 408 may include communication circuitry, such as, for example, a transceiver circuitry including an antenna and other communication media interfaces to connect to a wired and/or wireless communication protocol. The communication circuitry may, in at least some example embodiments, enable the transmission of data signals and/or reception of signals from other network entities, such as the at least one sensor 218, the first control unit 210, the second control unit 216, the user device 104, or other entities of FIG. 1.

In an embodiment, the processor 402 receives the data from at least the at least one sensor 218, the first control unit 210, the second control unit 216, and the user device 104 via a communication module (such as the communication module 408). The processor 402 is configured to render the digitized data on the application 116 via the network 112. Further, the processor 402 may transmit the signal to the first control unit 210 for operating the at least one radiating light source 212 to emit the radiating light of different wavelengths sequentially. Furthermore, the processor 402 may be configured to generate the digitized data based on processing the electrical signal received from the at least one sensor 218. The one or more operations performed by the first control unit 210, the second control unit 216, and the control circuitry 202 are explained with reference to FIG. 2, therefore they are not reiterated herein for the sake of brevity.

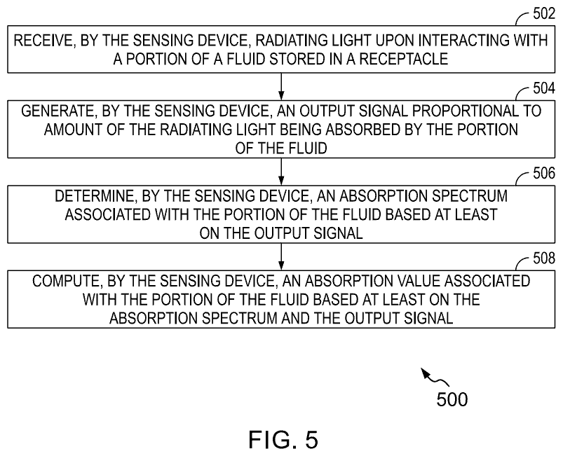

FIG. 5 illustrates a flow diagram 500 of a method for determining the aging of a portion of the fluid, in accordance with the embodiments of the present disclosure. The method 700 depicted in the flow diagram may be executed by, for example, the sensing device 110. Operations of the flow diagram of the method 500, and combinations of the operations in the flow diagram of the method 500, may be implemented by, for example, hardware, firmware, a processor, circuitry, and/or a different device associated with the execution of software that includes one or more computer program instructions.

Prior to the measurement process of determining the aging of the portion of the fluid, a plurality of preparatory operations (hereinafter interchangeably referred to as “preparatory operations”) are carried out to compensate for ambient parameters and to initialize the internal hardware components. The preparatory operations may include initializing internal hardware components such as General-Purpose Input Output (GPIO), Direct Memory Access (DMA), Universal Asynchronous Receiver-Transmitter (UART), Inter-Integrated Circuit (I2C), timers, Serial Peripheral Interface (SPI), Analog to Digital Converters (ADCs), Random Number Generator (RNGs), radio, and others that are associated with the sensing device 110.

Further, the preparatory operations may include loading the calibration values associated with the sensing device 110 from the memory associated with the sensing device 110. The calibration values may be provided by a manufacturer of the sensing device 110 or may be determined through preliminary experiments that may be performed prior to the process of determining the aging and the concentration of the alcohol. The sensing device 110 is configured to monitor the power supply, and when the power supply drops below a predefined value (For e.g., a voltage level below 3.4V may be indicated as a low voltage level), a low-battery condition is indicated while subsequent processes are performed. Furthermore, the sensing device 110 may be provided with a status Light Emitting Diode (LED) configured to indicate the operational status of the sensing device 110, offering temporary visual confirmation that the sensing device 110 is powered on and functioning correctly. The at least one sensor 218 associated with the sensing device 110 includes a temperature sensor, a liquid level capacitive sensor, a spectroscopy sensor (hereinafter the term “spectroscopy sensor” is interchangeably used as “photo detector”), and the like. The sensing device 110 supports a sequential configuration of the at least one sensor 218, where power is provided to each sensor of the at least one sensor 218 only during measurement and disabled once the measurement process is completed.

Further, the sensing device 110 is configured to measure ambient parameters associated with the receptacle to compensate for any background absorption, scattering losses, and optical characteristics associated with the receptacle 106a or the sensing device 110. The ambient parameters include ambient temperature and lighting that may be observed in the photo detector response over time. The sensing device 110 measures the photo detector response when the at least one radiating light source 212 is turned off to compensate for any ambient lighting and slight variation in photo detector response over time. Further, the at least one sensor 218 associated with the sensing device 110 may include separate temperature sensors to measure ambient temperature and the temperature associated with the portion of the fluid. For example, the sensing device 110 may be associated with a temperature sensor 1 configured to measure ambient air temperature and a temperature sensor 2 configured to measure the temperature associated with the portion of the fluid.

Thereafter, the power supply provided to the temperature sensors may be turned off. Further, the sensing device 110 may be configured to provide the power supply to the liquid level capacitive sensor, configured to measure the level of the portion of the fluid. Following that, the sensing device 110 acquires and processes the liquid capacitive sensor readings (measured level of the portion of the fluid in millimeters) and may turn off the power supply provided to the liquid capacitive sensor.

Following the measurements of the ambient parameters and the level of the portion of the fluid, the sensing device 110 is configured to emit the radiating light of the plurality of wavelengths through the portion of the fluid. Upon completion of the preparatory operations explained above, the sensing device 110 proceeds with the measurement of the concentration of the alcohol content and the aging of the portion of the fluid, as per the operations 502 to 508.

At operation 502, the method 500 includes receiving, by the sensing device 110, radiated light upon interacting with a portion of a fluid stored in the receptacle 106a. Upon receiving the radiating light, the sensing device 110 utilizes the at least one sensor to determine one or more parameters associated with the portion of the fluid based on the radiating light being detected by the at least one sensor 218 upon interacting with the portion of the fluid in the receptacle 106a. The one or more parameters associated with the portion of the fluid sample comprise at least one of temperature, fluid level, and humidity.

At operation 504, the method includes generating, by the sensing device 110, an output signal proportional to the amount of the radiating light being absorbed by the portion of the fluid. The output signal may further include at least one of an ambient temperature valid flag, a fluid temperature valid flag, fluid level measurement valid flag, spectroscopy measurement valid flag, low battery indication (when the power supply is below the predefined value), ambient temperature (in ° C.), temperature associated with the portion of the fluid (in ° C.), level of the portion of the fluid in millimeters, humidity in percent, liquid capacitive sensor readings, photo detector response, and the like. In an embodiment, the output signal may indicate the concentration of the alcohol content in the portion of the fluid in percent (%). In another embodiment, the control circuitry may be configured to compute the concentration of the alcohol content in the portion of the fluid based on the output signal.

At operation 506, the method includes determining, by the sensing device 110, an absorption spectrum associated with the portion of the fluid based at least on the output signal.

Based on the output signal, the sensing device 110 computes absorbance by comparing the radiating light being detected by the at least one sensor to the calibration values, using a logarithmic relationship defined by Beer-Lambert law. This calculation yields absorbance as a function of wave length, that is independent of the incident radiation intensity associated with the radiating light and directly related to the concentration of the alcohol under linear conditions. By sequentially scanning each wavelength across the plurality of wavelengths and repeating the photo detector readings (hereinafter the terms “photo detector readings” and “photo detector response” are used interchangeably) at each wavelength, a complete absorption spectrum is acquired. The resulting absorption spectrum, represented as absorbance versus wavelength, contains characteristic absorption features that correspond to specific electronic, vibrational, or rotational transitions of the portion of the fluid. Specifically, the absorbance associated with the near infrared region is utilized for determining the concentration of alcohol content in the portion of the fluid, the color and pH associated with the portion of the fluid, and the like. These spectral features are subsequently analyzed for qualitative identification of alcohol content and, when combined with calibration values, for quantitative determination of the concentration of the alcohol in the portion of the fluid.

At operation 508, the method includes computing, by the sensing device 110, the absorption value associated with the portion of the fluid based at least on the absorption spectrum and the output signal. The absorption value is indicative of the concentration of the alcohol content and an age factor associated with the portion of the fluid.

The sensing device 110 is configured to compute the absorption value by compensating for the ambient conditions and the sensing device 110 calibrations using the calibration values and the ambient parameters. Further, the sensing device 110 may be configured to compensate for changes in the temperature by utilizing a temperature compensation factor. The temperature compensation factor may be empirically determined. In one scenario, the temperature compensation factor may be empirically found to be 99.4 for a fluid with 50% alcohol content, using the sensing device 110. The temperature compensation factor may vary based on device to device variation and the concentration of the alcohol content. Hence, to compensate for the device to device variation, the sensing device 110 may be configured with an offset, Iᵢᵣ₀. An equation for temperature compensation may be provided as:

$${T}_{cf \_ do} = ( {I}_{i r 0} - 161 \; 99 ) / 800 \qquad ( 1 )$$

Where T_{cf_do} represents the temperature compensation factor, and Iᵢᵣ₀ represents the offset to compensate for device to device variation. Further, the photo detector response to the radiating light of near infrared range, compensated for temperature and device to device variation, may be represented as,

$${I}_{ir \_ tc 1} = {I}_{i r} + {T}_{cf 1} * ( T - T 0 ) \qquad ( 2 )$$

Furthermore, to account for variation due to the concentration of the alcohol content, the ethanol estimate, Ethᵢ, may be defined using a second-order polynomial whose coefficients are empirically established. Further, the photo detector response to the radiating light of the near infrared range, compensated for temperature and variation due to the concentration of the alcohol content, may be represented as,

$${I}_{ir \_ tc} = {I}_{i r} + {T}_{cf 2} * ( T - T 0 ) \qquad ( 3 )$$

The sensing device 110 may be further configured to compensate for ambient lighting, by defining an ideal value of photo detector response to no ambient lighting, established by sampling a number of sensing devices and computing an average of the results. The equation for the photo detector response that compensates for the ambient lighting may be given as,

$${I}_{ir \_ c} = {I}_{ir \_ tc} + ( {I}_{a i} - {I}_{a} ) , \qquad ( 4 )$$

where I_{ir_c} represents the photo detector response that compensates for ambient lighting, I_{ir_tc} represents the temperature-compensated photo detector response, Iₐᵢ represents the ideal value of the photo detector response for no ambient lighting, and Iₐ represents the photo detector response at ambient lighting conditions.

Further, the photo detector response is normalized to account for device variations. The normalized photo detector response is given as,

$${I}_{ir \_ nom} = {I}_{ir \_ c} / {I}_{ir 0}$$

Further, the sensing device 110 is configured to estimate the concentration of the alcohol content in the portion of the fluid by compensating for the ambient parameters, using first-order polynomial coefficients, established empirically, by using the following equation,

$$Eth = 108 + ( 692 * {I}_{ir \_ 1} ) \qquad ( 5 )$$

Where the I_{ir_1} represents the photo detector response obtained using the following equation,

$${I}_{ir \_ 1} = log ( {I}_{ir \_ norm} ) \qquad ( 6 )$$

Further, the sensing device 110 is configured to determine the color associated with the portion of the fluid based on the selective absorption of the radiating light by molecules associated with the portion of the fluid at a specific wavelength. When the radiating light passes through or is reflected from the portion of the fluid, the extent of the absorption is determined based on the photo detector response at multiple color wavelengths (such as red, green, and blue) governed by Beer-Lambert's law, A=εbc, where absorbance (A) is directly proportional to the molar absorptivity (ε), path length (b), and concentration (c) of the absorbing species (concentration of the alcohol content). Ratio of the photo detector response to intensity of the radiating light transmitted by the at least one radiating light source 212 indicates the relative intensity of the radiating light detected at each wavelength after interaction with the portion of the fluid. These ratios are then compared with the calibrated values to identify which wavelengths are absorbed or least transmitted. The wavelength region with the minimum photo detector response corresponds to maximum absorption, and the observed color of the portion of the fluid is determined as the complementary color of the absorbed wavelength. Thus, calibration values ensure accurate sensor scaling, while the photo detector response ratios provide the spectral balance needed to objectively determine the color of the portion of the fluid. Ratio of photo detector response to intensity of the radiating light transmitted by the at least one radiating light source 212, in addition to the calibration value, may be utilized in a look-up table to determine lightness or darkness of the solution. The absorption indicates the extent of the interaction between the charred layer of wood inside the barrel and the portion of the fluid over time. By comparing the extent of absorption over time, a comparative evaluation of how fast or how slowly the aging of the portion of the fluid is taking place with respect to fluid stored in other barrels.

Upon successful completion of the computation of the concentration of the alcohol content in the portion of the fluid, the sensing device 110 may be configured to shut down for a predefined time, by turning off the power supply to the at least one sensor and deactivates internal hardware components for the predefined time. Furthermore, the sensing device 110 may be configured to turn on after the predefined time.

Various embodiments of the disclosure, as discussed above, may be practiced with steps and/or operations in a different order, and/or with hardware elements in configurations, which are different than those which are disclosed. Therefore, although the disclosure has been described based on these exemplary embodiments, it is noted that certain modifications, variations, and alternative constructions may be apparent and well within the spirit and scope of the disclosure.

1. A sensing device comprising:
a housing,
at least one radiating light source disposed within the housing, the at least one radiating light source configured to emit radiating light of a plurality of wave lengths through a portion of a fluid stored in a receptacle,
at least one sensor configured to receive the radiating light upon interacting with the portion of the fluid in the receptacle and generate an output signal proportional to the amount of the radiating light being absorbed by the portion of the fluid, and
a control circuitry communicably coupled to the at least one sensor and the at least one radiating light source, the control circuitry configured, at least in part, to:
receive the output signal from the at least one sensor,
determine an absorption spectrum associated with the portion of the fluid based at least on the output signal, wherein the absorption spectrum is indicative of variation of absorption associated with the portion of the fluid across a plurality of wavelengths, and
determine a rate of aging based on the absorption spectrum.

2. The sensing device as claimed in claim 1, wherein the sensing device is further caused to:
detect ambient parameters of the receptacle, wherein the ambient parameters comprise at least one of temperature and lighting;
determine one or more parameters associated with the portion of the fluid based on the radiating light being detected by the at least one sensor upon interacting with the portion of the fluid in the receptacle, wherein the one or more parameters associated with the portion of the fluid comprise at least one of temperature, fluid level, and humidity;
determine color associated with the portion of the fluid based at least on the absorption spectrum; and
compute an absorption value associated with the portion of the fluid based at least on the absorption spectrum and the one or more parameters associated with the portion of the fluid, wherein the absorption value associated with the portion of the fluid is indicative of concentration of alcohol content associated with the portion of the fluid.

3. The sensing device as claimed in claim 1, further comprising:
a base structure configured to support a right support structure and a left support structure, wherein the base structure, the left support structure, and the right support structure collectively form the housing;
the right support structure extending from the base structure, the right support structure configured to enclose the at least one radiating light source; and
the left support structure configured to enclose the at least one sensor,
wherein the left support structure and the right support structure are positioned at a distance.

4. The sensing device as claimed in claim 1, further comprising:
a first control unit disposed in the right support structure communicably coupled to the at least one radiating light source, wherein the first control unit is configured to provide power supply to the at least one radiating light source; and
a second control unit disposed in the left support structure communicably coupled to the at least one sensor, wherein the second control unit is configured to perform at least one of:
provide the power supply to the at least one sensor,
amplify the output signal to generate an amplified output signal,
convert the amplified output signal into discrete digital values, and
transmit the discrete digital values to the control circuitry, wherein the discrete digital values correspond to intensity levels of the radiating light associated with at least one of a specific time interval or a specific wavelength.

5. The sensing device as claimed in claim 1, further comprising:
a first window associated with the right support structure configured to perform effective transmission of the radiating light from the at least one radiating light source to the at least one sensor; and
a second window associated with the left support structure configured in line with the right support structure, wherein the second window is configured to receive the radiating light and direct the received radiating light to the sensor circuitry.

6. The sensing device as claimed in claim 1, further comprising:
an enclosure removably coupled to the housing, the enclosure configured to enclose the control circuitry and a power source.

7. The sensing device as claimed in claim 2, wherein the sensing device is further caused to:
render the absorption value and the color associated with the portion of the fluid to one or more users upon computing the absorption value and determining the color associated with the portion of the fluid.

8. A method performed by a sensing device, comprising:
receiving radiating light upon interacting with a portion of a fluid stored in a receptacle;
generating an output signal proportional to amount of the radiating light being absorbed by the portion of the fluid;
determining an absorption spectrum associated with the portion of the fluid based at least on the output signal, wherein the absorption spectrum is indicative of variation of absorption associated with the portion of the fluid across a plurality of wavelengths; and
determining a rate of aging based on the absorption spectrum.

9. The method as claimed in claim 8, further comprising:
detecting ambient parameters of the receptacle, wherein the ambient parameters comprise at least one of temperature and lighting;
determining one or more parameters associated with the portion of the fluid based on the radiating light being detected by the at least one sensor upon interacting with the portion of the fluid in the receptacle, wherein the one or more parameters associated with the portion of the fluid comprise at least one of temperature, fluid level, and humidity;
determining color associated with the portion of the fluid based at least on the absorption spectrum; and
computing an absorption value associated with the portion of the fluid based at least on the absorption spectrum and the one or more parameters associated with the portion of the fluid, wherein the absorption value associated with the portion of the fluid is indicative of concentration of alcohol content associated with the portion of the fluid.

10. The method as claimed in claim 8, further comprising:
amplifying the output signal to generate an amplified output signal; and
converting the amplified output signal into discrete digital values, wherein the discrete digital values correspond to intensity levels of the radiating light associated with at least one of a specific time interval or a specific wavelength.

11. The method as claimed in claim 9, further comprising:
rendering the absorption value and the color associated with the portion of the fluid to one or more users upon computing the absorption value and determining the color associated with the portion of the fluid.

12. A system comprising:
a receptacle configured to store a portion of a fluid; and
a sensing device disposed within the receptacle, the sensing device comprising:
a housing,
at least one radiating light source disposed within the housing, the at least one radiating light source configured to emit radiating light of a plurality of wavelengths through the portion of the fluid in the receptacle,
at least one sensor configured to receive the radiating light upon interacting with the portion of the fluid in the receptacle and generate an output signal proportional to the amount of the radiating light being absorbed by the portion of the fluid, and
a control circuitry communicably coupled to the at least one sensor and the at least one radiating light source, the control circuitry configured, at least in part, to:
receive the output signal from the at least one sensor,
determine an absorption spectrum associated with the portion of the fluid based at least on the output signal, wherein the absorption spectrum is indicative of variation of absorption associated with the portion of the fluid across a plurality of wavelengths, and
determine a rate of aging based on the absorption spectrum.

13. The system as claimed in claim 12, wherein the sensing device is further configured to:
detect ambient parameters of the receptacle, wherein the ambient parameters comprise at least one of temperature and lighting;
determine one or more parameters associated with the portion of the fluid based on the radiating light being detected by the at least one sensor upon interacting with the portion of the fluid in the receptacle, wherein the one or more parameters associated with the portion of the fluid comprise at least one of temperature, fluid level, and humidity;
determine color associated with the portion of the fluid based at least on the absorption spectrum; and
compute an absorption value associated with the portion of the fluid based at least on the absorption spectrum and the one or more parameters associated with the portion of the fluid, wherein the absorption value associated with the portion of the fluid is indicative of concentration of alcohol content associated with the portion of the fluid.

14. The system as claimed in claim 12, wherein the sensing device further comprises:
a base structure configured to support a left support structure and a right support structure, wherein the base structure, the left support structure, and the right support structure collectively form the housing;
the right support structure extending from the base structure, the right support structure configured to enclose the at least one radiating light source; and
the left support structure configured to enclose the at least one sensor,
wherein the left support structure and the right support structure are positioned at a distance.

15. The system as claimed in claim 12, wherein the sensing device further comprises:
a first control unit disposed in the right support structure communicably coupled to the at least one radiating light source, wherein the first control unit is configured to provide power supply to the at least one radiating light source; and
a second control unit disposed in the left support structure communicably coupled to the at least one sensor, wherein the second control unit is configured to perform at least one of:
provide the power supply to the at least one sensor,
amplify the output signal to generate an amplified output signal,
convert the amplified output signal into discrete digital values, and
transmit the discrete digital values to the control circuitry, wherein the discrete digital values correspond to intensity levels of the radiating light associated with at least one of a specific time interval or a specific wavelength.

16. The system as claimed in claim 12, wherein the sensing device further comprises:
a first window associated with the right support structure configured to perform effective transmission of the radiating light from the at least one radiating light source to the at least one sensor; and
a second window associated with the left support structure configured in line with the right support structure, wherein the second window is configured to receive the radiating light and direct the received radiating light to the sensor circuitry.

17. The system as claimed in claim 12, wherein the sensing device further comprises:
an enclosure removably coupled to the housing, the enclosure configured to enclose the control circuitry and a power source.

18. The system as claimed in claim 13, wherein the sensing device is further configured to:
render the absorption value and the color associated with the portion of the fluid to one or more users upon computing the absorption value and determining the color associated with the portion of the fluid.
